(19) United States
(12) Patent Application Publication
(10) Pub. No.: US 2007/0202521 A1
(43) Pub. Date: Aug. 30, 2007

(54) Single Molecule DNA Sequencing Using Fret Based Dynamic Labeling

(75) Inventor: Hongye Sun, Belmont, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/675,032

(22) Filed: Feb. 14, 2007

Related U.S. Application Data
(60) Provisional application No. 60/773,619, filed on Feb. 14, 2006.

(51) Int. Cl. C12Q 1/68; C07H 21/04; C12M 3/00

(52) U.S. Cl. 435/6; 435/287.2; 536/25.32

(57) ABSTRACT

Some of the subject methods may be used to sequence single molecules of polynucleotides of interest. The methods of the invention employ fluorescent intercalators as a donor in FRET (fluorescence resonance energy transfer). Fluorescent intercalators intercalate within the double-stranded region of the primed template. The intercalators may be used as donors in FRET. Additional molecules of the fluorescent intercalator may be incorporated into the newly formed double-stranded regions that are formed as the primer is extended.

### CROSS-REFERENCE TO RELATED APPLICATIONS

This application claims a priority benefit under 35 U.S.C. § 119(e) from U.S. Provisional Application No. 60/773,619 filed Feb. 14, 2006, which is incorporated herein by reference.

### FIELD OF THE INVENTION

The Invention is in the field of DNA sequencing.

### BACKGROUND

It is of interest to provide methods of determining the base sequence of polynucleotides such as DNA and RNA. Conventional methods of DNA sequencing, such as Sanger sequencing using electrophoresis, employ number of identical polynucleotides in order to produce detectable signals. An alternative approach is to determine the sequence of individual polynucleotides of a single molecule. The subject invention employs Forster energy transfer between a donor and quencher (acceptor) fluorophore to detect polymerase-mediated incorporation of nucleotides into a primer.

### EMBODIMENTS OF THE INVENTIONS

The methods of the invention employ fluorescent intercalators as a donor in FRET (fluorescence resonance energy transfer) for use in nucleic acid sequencing reactions. By employing fluorescent intercalators as donor in FRET, the photobleaching of the donors may be reduced because the same molecule is not repeatedly exposed to an excitation source. The fluorescent intercalators intercalate within the double-stranded region of the primed template. The intercalators may be used as donors in FRET. Additional molecules of the fluorescent intercalator may be incorporated into the newly formed double-stranded regions that are formed as the primer is extended. The intercalator molecules may be present in free solution form prior to intercalation.

The nucleotide sequence information generated from a primed template may be one or more bases in length. Single base determination may be used for the identification of single nucleotide polymorphisms (SNPs).

One embodiment of the invention is a method of determining the base sequence of a polynucleotide of interest. A complex is formed between a DNA polymerase and a primed template. Either the DNA polymerase, the template, or the primer, can be immobilized on the surface of a solid support. FRET between the donor and acceptor is only efficient when the dyes are in close proximity to one another. Excitation of the acceptor dye (quenching of the signal from the donor) can then be detected so as to be indicative of the incorporation of the labeled nucleotide. In some embodiments of the invention (e.g. nucleotides that are labeled with a fluorophore at the gamma phosphate position), the acceptor fluorophore is released upon incorporation of the labeled nucleotide into the extending primer, there by producing a detectable signal as FRET is interrupted. Detection of the FRET signal produced upon incorporation of the dye labeled nucleotide may be coupled with the loss of energy transfer from diffusion of the gamma-labeled phosphate so as to provide greater certainty in detecting actual incorporation events.

The subject methods may be applied to sequence individual polynucleotides. Multiple complexes formed between the DNA polymerase and a primed template may be analyzed in parallel on different regions of the same solid support. The signals from multiple primed templates analyzed in parallel may be combined for analysis (typically mediated by a computer) so as to reduce uncertainty associated with the identity of nucleotide base at a given position in the sequence of interest.

Fluorescent dye labeled nucleotides (dNTPs) employed in the subject methods can be labeled with different acceptor fluorophores that may be distinguishable from one another based on emission spectra. The identity of the specific fluorophore can be correlated with the identity of the specific nucleotide base (A, C, G, T, or analogs thereof) so as to provide for the identification of the base on the incorporated nucleotide.

The fluorescent dye labeled nucleotides may optionally be reversibly blocked at the 3′ or 2′ sugar position in some embodiments so as to act as extension reaction terminators

The intercalating dye employed in the subject methods acts a donor in a FRET reaction. The intercalating dyes intercalate into double-stranded polynucleotides. The intercalating dye is fluorescent. The intercalating dye may be a fluorescent dye or may be fluorescent dye conjugated to a molecule that is primarily an intercalator. Intercalating dyes are well known to the person of ordinary skill in the art. Examples of intercalating dyes include, but are not limited to, phenanthridines and acridines, such as ethidium bromide, propidium iodidem, hexidium iodide, dihydroethidium, ethidium homodimers, acridine orange, 9-amino-6-chloro-2-methoxyacridine; indoles and imaidazoles such as DAPI, bisbenzimide dyes, Actinomycin D, Nissl stains, hydroxystilbamidine: SYBR Green™ (Molecular Probes). Many fluorescent dyes are commercially available.

A wide variety of fluorescent dyes may be used as quenchers (acceptors). The choice of suitable quenchers will be a function of the choice of the donor fluorophore used as the intercalator as the excitation wavelength should be able to support the desired FRET.

The DNA polymerase may be any enzyme having DNA polymerase activity, including enzymes that are not typically characterized as a DNA polymerases, e.g., a reverse transcriptase. Suitable polymerase may be thermostable or not thermostable. It of interest to provide polymerases that are resistant to denaturation by the conditions employed in the method. It is also of interest to use DNA polymerases that are highly processive such phi 29 and other DNA polymerases with similar processivity properties, e.g. see U.S. Pat. No. 5,576,204.

General guidance on practicing aspects of this invention, including detection of fluorescence of single fluorophore molecules can be found in PCT patent application WO 02/04680 A2, entitled “Real time sequence determination”, which describes methods of sequencing single DNA molecules using immobilized DNA polymerases or templates, wherein fluorescence energy transfer between donor fluorophore on a polymerase and acceptors fluorophores on dNTPs are detected. Additional guidance can be found in PCT patent application WO 01/16375 A2, entitled “High Speed Parallel Molecular Nucleic Acid Sequencing.” Further guidance on imaging can be found in U.S. Pat. Nos. 7,056,676; 7,056,661; 7,052,847; 7,033,764; and 7,118,907. The aforementioned patents and patent applications are hereby incorporated by reference

Although the invention has been described in detail for the purposes of illustration, it is understood that such detail is solely for that purpose, and variations can be made therein by those skilled in the art without departing from the spirit and scope of the invention which is defined by the following claims.

What is claimed is:

1. A method sequencing a polynucleotide comprising the steps,
providing an immobilized primed polynucleotide template DNA polymerase complex, wherein said complex comprises a primer strand and an intercalating dye that is a donor in a FRET reaction incorporating a fluorescent dye labeled nucleotide into the primer strand, wherein
the fluorescent dye is an acceptor in a FRET reaction,
detecting energy transfer event between the intercalating dye and the dye on the labeled nucleotide, wherein the energy transfer event is indicative of the incorporation of the nucleotide into the primer strand.

2. A method according to claim 1, wherein the DNA is immobilized on a solid support.

3. The method of claim 1, wherein the DNA template is immobilized on a solid support.

4. The method of claim 1, wherein the primer strand is immobilized on a sold support.

5. The method of claim 1, wherein the polymerase is mixed with 4 different nucleotide, each nucleotide being labeled with a different fluorescent dye.

6. The method of claim 1, wherein the intercalating dye comprises a fluorophore conjugated to a fluorophore.

7. The method of claim 1, wherein the intercalating dye is fluorescent dye.

8. The method of claim 1, wherein the fluorescent dye on the fluorescent dye-labeled nucleotide is joined at a position on a base in the nucleotide.

9. The method of claim 1, wherein the fluorescent dye on the fluorescent dye-labeled nucleotide is joined at a position on a phosphate in the nucleotide.

10. The method of claim 9, wherein the fluorescent dye is attached to the gamma phosphate position.
